(19) United States
(12) Patent Application Publication
(10) Pub. No.: US 2020/0093647 A1
(43) Pub. Date: Mar. 26, 2020

(54) Heel Repair Dressing

(71) Applicant: Glenn McClendon, Conway, AR (US)

(72) Inventor: Glenn McClendon, Conway, AR (US)

(21) Appl. No.: 16/138,330

(22) Filed: Sep. 21, 2018

(51) Int. Cl. A61F 13/06; A61M 35/00; A61F 13/00

(52) U.S. Cl. CPC A61F 13/067; A61F 13/00068; A61F 13/00063; A61M 35/00

(57) ABSTRACT

A device designed for exfoliating, soothing, and healing damages skin on the heel of the foot. The device is designed to keep therapeutic substances in direct contact the skin of the foot and allow it to remain in place for a desired amount of time. The device is adaptable to a multitude of therapeutic substances as well as feet of varying sizes.

### FIELD OF THE INVENTION

The present invention relates generally to medical dressings for human skin. More particularly, the present invention relates to dressings which repair or restore damaged skin on the human foot

### BACKGROUND OF THE INVENTION

Dry and cracked skin on the heel of the foot is and has been an issue for many people for quite some time. For many people the issue is merely cosmetic, but for others, pain will accompany the damaged skin. But, because the heel of the foot is heavily used in everyday living, it is difficult to apply any remedial products which will stay on the heel for a long enough time to have any noticeable effect. As a result, it is desirable to provide a product which will protect the heel while keeping a medicated product in contact with the skin for a time sufficient to allow the skin to heal.

### SUMMARY OF THE INVENTION

The present invention is a dressing for exfoliating, soothing, and healing damaged skin on the heel of the foot. The dressing consists of a shaped bandage configured to contour to the user's heel and products which keep therapeutic materials in contact with the skin which serve to remediate the damaged skin.

The object of the present invention is to provide a product which will reliably keep therapeutic and/or medicated products in direct contact with the skin of the heel while, at the same time, protecting the heel for several hours at a time while the skin is allowed to heal.

It is a further object of this invention to provide a product which is adaptable to heels of varying sizes such that users with differently sized feet may use the device.

The advantages of the present invention include that it is adaptable to varying heel sizes as well as varying medicated or therapeutic products and is simple to manufacture and assemble.

These and other objects and advantages of the present invention, along with features of novelty appurtenant thereto, will appear or become apparent in the course of the following descriptive sections.

### BRIEF DESCRIPTION OF THE DRAWINGS

In the following drawings, which form a part of the specification and which are to be construed in conjunction therewith, and in which like reference numerals have been employed throughout wherever possible to indicate like parts in the various views:

FIG. 1 is a top isometric view of the heel dressing of a first embodiment of the present invention;

FIG. 2 is a frontal view of the heel dressing of a first embodiment of the present invention;

FIG. 3 is a bottom isometric view of the heel dressing of a first embodiment of the present invention; and

FIG. 4 is a top isometric view of the heel dressing of a second embodiment of the present invention.

### DETAILED DESCRIPTION OF THE PREFERRED EMBODIMENTS

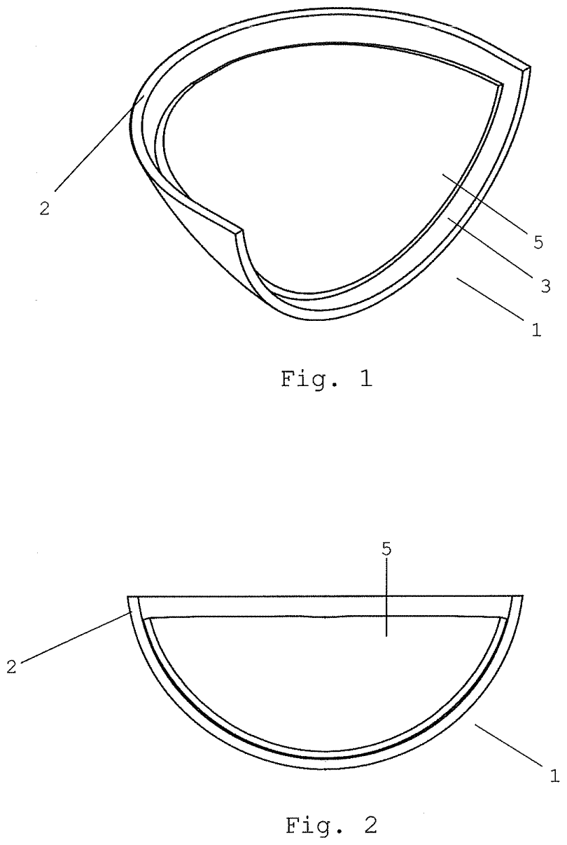

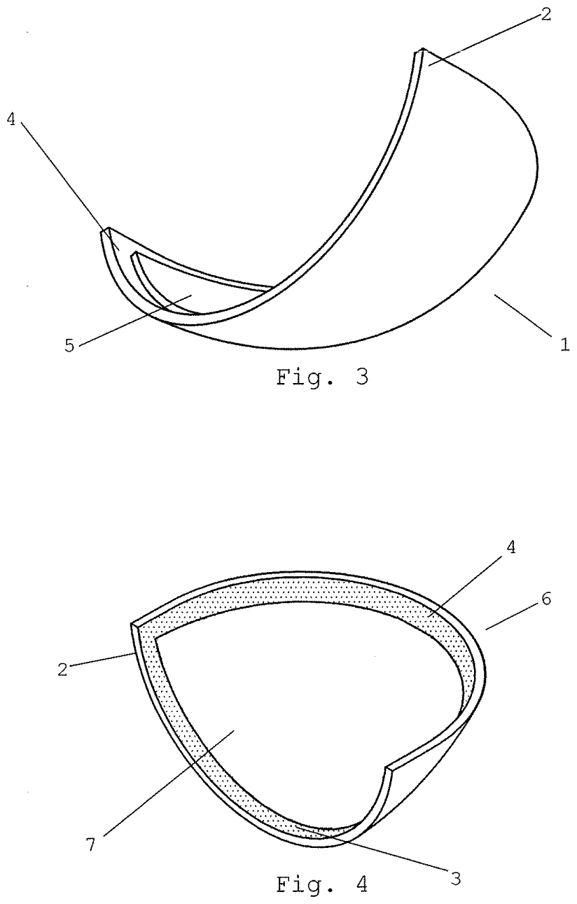

Referring to the drawings, FIGS. 1-3 depict a first embodiment of a heel dressing 1 featuring a cup 2 and one or more treatment pads 4.

The cup 2 shall be configured generally to mirror the overall shape of a human heel. The shape shall roughly resemble a quarter-spheroid however it may deviate from the proportions of a perfect quarter-sphere. The cup 2 shall have a concave inner portion 3 into which the heel of the foot is placed. The cup is preferably constructed of a flexible, semi rigid material such that it may flex in its operation in affixing the cup to the user's foot as well as with the movement of the user in daily life. The preferred material of the cup is a shape-molded fabric, but various flexible polymers, rubbers, or other materials known in the art may be used. The cup may also have one or more adhesive areas 4 configured to removably attach the dressing to the user's heel.

The treatment pad 5 is located in the concave inner portion 3 of the cup and is configured to be placed in direct contact with the user's heel. The treatment pad 4 may be affixed to the cup 2. The treatment pad 5 may be hydrogel or other similar dressings known in the art. The treatment pad may also be an absorbent material such as foam, gauze, or other known absorbent materials. The treatment pad may be dry or it may be saturated with a moisturizing, keratolytic, exfoliating, or anti-inflammatory agent or other mediated agent known in the art.

To use the heel dressing 1, a user shall take the dressing 1 and orient it such that the concave portion 3 of the cup 2 and the treatment pad 5 is facing upward. If the treatment pad comprises a hydrogel or similar material, or if the treatment pad comprises an absorbent material saturated with a medicated agent, the user will place his or her heel into the cup such that the heel makes direct contact with the treatment pad. The user then attaches the dressing to the foot via the adhesive 4 if available or via tape or compressive piece such as a wrap bandage or sock. If the dressing utilizes an absorbent treatment pad which has not been saturated with a medicated agent, the user may apply a medicated agent of his or her choosing or use the dressing with none.

Referring to the drawings, FIG. 4 depicts a second embodiment 6 of a heel dressing featuring a cup 2 and a recess 7. The cup 2 of the second embodiment is the same as the cup 2 described in relation to the first embodiment of the heel dressing 1 above.

The recess 7 is located on the surface of concave portion 3 of the cup 2. The recess 6 may shall be an area in the concave portion 3 of the cup 2 that is recessed into the surface of the cup or surrounded by a raised portion. Ultimately, the recess 7 shall be configured to house and partially contain a treating agent applied by the user.

To use the second embodiment 6 of the heel dressing, a user shall take the dressing 6 and orient it such that the concave portion 3 of the cup 2 and the recess 7 is facing upward. The user may apply a medicated agent of his or her choosing into the recess and place his or her heel into the cup such that the heel makes direct contact with the medicated agent located in the recess. The user then attaches the dressing to the foot via the adhesive 4 if available or via tape or compressive piece such as a wrap bandage or sock.

What is claimed is:

1. A heel dressing comprising:
a cup configured to be removably attached to a human heel and having the general shape of a quarter-spheroid;
said cup having a concave inner portion; and
a treatment pad configured to be placed in direct contact with the user's heel.

2. The heel dressing as described in claim 1 further comprising an adhesive area on the concave inner portion of the cup.

3. The heel dressing as described in claim 1 wherein the treatment pad comprises hydrogel.

4. The heel dressing as described in claim 2 wherein the treatment pad comprises hydrogel.

5. The heel dressing as described in claim 1 wherein the treatment pad comprises an absorbent material saturated with a medicated agent.

6. The heel dressing as described in claim 2 wherein the treatment pad comprises an absorbent material saturated with a medicated agent.

7. The heel dressing as described in claim 1 wherein the treatment pad comprises a dry absorbent material.

8. The heel dressing as described in claim 2 wherein the treatment pad comprises a dry absorbent material.

9. A heel dressing comprising:
a cup configured to be removably attached to a human heel and having the general shape of a quarter-spheroid;
said cup having a concave inner portion with a recess configured to contain a medicated treating agent.
a treatment pad configured to be placed in direct contact with the user's heel.

10. The heel dressing as described in claim 9 further comprising an adhesive area on the concave inner portion of the cup.
